(19) United States
(12) Patent Application Publication
(10) Pub. No.: US 2023/0082779 A1
(43) Pub. Date: Mar. 16, 2023

(54) MEDICAL IMAGE PROCESSING DEVICE, OPERATION METHOD OF MEDICAL IMAGE PROCESSING DEVICE, AND NON-TRANSITORY COMPUTER READABLE MEDIUM

(71) Applicant: FUJIFILM Corporation, Tokyo (JP)

(72) Inventor: Shumpei KAMON, Kanagawa (JP)

(73) Assignee: FUJIFILM Corporation, Tokyo (JP)

(21) Appl. No.: 17/932,035

(22) Filed: Sep. 14, 2022

(30) Foreign Application Priority Data
Sep. 16, 2021 (JP) 2021-150905

(51) Int. Cl. G06T 7/00; G16H 30/40; G16H 30/20

(52) U.S. Cl. CPC G06T 7/0012; G16H 30/40; G16H 30/20

(57) ABSTRACT

A medical image processing device performs recognition processing on an acquired medical image, performs a control of displaying, on a display, the medical image and a result of the recognition processing of the medical image, receives an evaluation related to the result of the recognition processing from a user based on the displayed medical image and the displayed result of the recognition processing, determines whether or not to store the medical image, which is a target of the evaluation, in a data storage unit based on the evaluation, and performs a control of storing, in the data storage unit, the medical image determined to be stored.

### CROSS-REFERENCE TO RELATED APPLICATIONS

This application claims priority under 35 U.S.C § 119(a) to Japanese Patent Application No. 2021-150905 filed on 16 Sep. 2021. The above application is hereby expressly incorporated by reference, in its entirety, into the present application.

### BACKGROUND OF THE INVENTION

### 1. Field of the Invention

The present invention relates to a medical image processing device, an operation method of a medical image processing device, and a non-transitory computer readable medium.

### 2. Description of the Related Art

In the medical field, diagnosis support information for supporting a diagnosis of a doctor is obtained by performing image recognition processing using a medical image obtained by various modalities, such as an endoscope, a computed tomography (CT), or magnetic resonance imaging (MRI). In recent years, various methods of obtaining desired information by the image recognition processing using a machine learning technique have been developed.

In a case in which image recognition processing of a medical image is performed using machine learning technique, a large amount of appropriate medical image data is required in order to handle various clinical conditions. As a device that acquires a medical image during the image recognition processing for appropriate medical image data, there is known a medical image processing device that acquires a user input signal by a freeze button and the like, and selects the medical image from the medical image for which a result of the recognition processing related to a region-of-interest is obtained, in a case in which the user input signal is acquired (JP2021-045337A, corresponding to US2021/082568A1). In addition, there is known a medical image processing device that stores a medical image after receiving correction of an analysis result with respect to the analysis result of the medical image (WO2019/008941A, corresponding to US2020/143936A1).

### SUMMARY OF THE INVENTION

In a case in which image recognition processing using machine learning technique is performed, in a case in which a learning model constructed by supervised learning is used, it is preferable to use a learning model constructed by being trained using a large number of training data useful for learning. Therefore, in order to perform the image recognition processing of the medical image with high accuracy, it is preferable to use the learning model that has been trained using a large number of medical images useful for learning as training data. However, it takes a lot of effort to collect a large number of medical images useful for learning.

The present invention is to provide a medical image processing device, an operation method of a medical image processing device, and a non-transitory computer readable medium for storing a computer-executable program which are capable of selectively accumulating medical images useful for learning.

An aspect of the present invention relates to a medical image processing device comprising a processor, in which the processor acquires a medical image in which a subject is reflected, performs recognition processing on the medical image, performs a control of displaying, on a display, the medical image and a result of the recognition processing of the medical image, receives an evaluation related to the result of the recognition processing from a user based on the displayed medical image and the displayed result of the recognition processing of the medical image, determines whether or not to store the medical image, which is a target of the evaluation, in a data storage unit based on the evaluation, and performs a control of storing, in the data storage unit, the medical image determined to be stored.

It is preferable that the processor perform a control of displaying, on the display, an instruction display prompting the user for the evaluation, and receive the evaluation after the instruction display is displayed.

It is preferable that the processor acquire the medical image during examination, and perform a control of displaying the instruction display at a preset timing after the examination ends.

It is preferable that the processor acquire the medical image during examination, perform a control of displaying, on the display, the medical image and the result of the recognition processing of the medical image at a preset timing after the examination ends, and receive the evaluation after the medical image and the result of the recognition processing of the medical image are displayed on the display.

It is preferable that the processor make the display for which the control of displaying the medical image and the result of the recognition processing of the medical image is performed during the examination, and the display for which the control of displaying the medical image and the result of the recognition processing of the medical image is performed after the examination ends different from each other.

It is preferable that the evaluation be performed based on an evaluation value indicating a degree to which the result of the recognition processing is a correct answer, and, in a case in which the evaluation value is within a preset range indicating that the degree to which the result of the recognition processing is the correct answer is low, the processor determine to store the medical image in the data storage unit.

It is preferable that the processor perform a control of temporarily storing the medical image in a temporary storage unit.

It is preferable that, in a case in which it is determined to store the medical image, the processor store the medical image stored in the temporary storage unit in the data storage unit.

It is preferable that, in a case in which it is determined to store the medical image, the processor perform a control of extracting a part of the medical image from the medical image stored in the temporary storage unit, and then storing the extracted part of the medical image in the data storage unit.

It is preferable that, in a case in which the result of the recognition processing of the medical image includes a preset specific content, the processor perform a control of storing, in the data storage unit, the medical image acquired in a preset period including a time point at which the medical image, which is a target of the recognition processing of the medical image, is acquired.

It is preferable that the processor receive diagnosis information related to a diagnosis made by the user on the subject, and perform a control of storing, in the data storage unit, the medical image and the diagnosis information in association with each other.

It is preferable that the processor perform a control of extracting the medical image in accordance with a content of the diagnosis information, and then storing the extracted medical image in the data storage unit.

It is preferable that, in a case in which the medical image includes individual information related to an individual having the subject, the processor perform a control of deleting the individual information from the medical image, and then storing the medical image in the data storage unit.

It is preferable that the processor perform a control of storing, in the data storage unit, the medical image and the result of the recognition processing of the medical image in association with each other.

It is preferable that the processor perform a control of storing, in the data storage unit, the medical image as a still picture and/or a motion picture including the medical image.

It is preferable that the processor make the display for which the control of displaying the instruction display is performed, the display for which the control of displaying the medical image is performed, and the display for which the control of displaying the result of the recognition processing of the medical image is performed different from each other, or make at least two thereof the same.

In addition, another aspect of the present invention relates to an operation method of a medical image processing device, the method comprising a step of acquiring a medical image in which a subject is reflected, a step of performing recognition processing on the medical image, a step of performing a control of displaying, on a display, the medical image and a result of the recognition processing of the medical image, a step of receiving an evaluation related to the result of the recognition processing from a user based on the displayed medical image and the displayed result of the recognition processing of the medical image, a step of determining whether or not to store the medical image, which is a target of the evaluation, in a data storage unit based on the evaluation, and a step of performing a control of storing, in the data storage unit, the medical image determined to be stored.

In addition, still another aspect of the present invention relates to a non-transitory computer readable medium for storing a computer-executable program causing a computer to execute a process of acquiring a medical image in which a subject is reflected, a process of performing recognition processing on the medical image, a process of performing a control of displaying, on a display, the medical image and a result of the recognition processing of the medical image, a process of receiving an evaluation related to the result of the recognition processing from a user based on the displayed medical image and the displayed result of the recognition processing of the medical image, a process of determining whether or not to store the medical image, which is a target of the evaluation, in a data storage unit based on the evaluation, and a process of performing a control of storing, in the data storage unit, the medical image determined to be stored.

According to the present invention, the medical images useful for learning can be selectively accumulated.

### BRIEF DESCRIPTION OF THE DRAWINGS

FIG. 1 is a block diagram showing a function of a medical image processing device.

FIG. 2 is a block diagram showing a configuration of the medical image processing device.

FIG. 3 is a block diagram showing a function of a recognition processing unit.

FIG. 4A is an explanatory diagram for describing processing of a detector that outputs a region of a detection region-of-interest, and FIG. 4B is an explanatory diagram for describing processing of a detector that outputs a rectangle indicating a position of the detection region-of-interest.

FIG. 5 is an image diagram displaying an endoscopic image and a result of recognition processing which is detection processing.

FIG. 6 is an image diagram displaying an endoscopic image and a result of recognition processing which is classification processing.

FIG. 7 is an image diagram displaying an endoscopic image and a result of recognition processing which is site recognition processing.

(a) of FIG. 8 is an image diagram during an examination, and (b) of FIG. 8 is an image diagram after the examination in which an instruction display is displayed ends.

(a) of FIG. 9 is an image diagram during an examination in which the result of the recognition processing is displayed, and (b) of FIG. 9 is an image diagram after the examination in which the result of the recognition processing and the instruction display are displayed ends.

FIG. 10 is an image diagram after the end of the examination in which the results of a plurality of pieces of recognition processing and the instruction display are displayed ends.

FIG. 11A is an image diagram of an instruction display having an instruction input frame, FIG. 11B is an image diagram of an instruction display having an evaluation value input button, FIG. 11C is an image diagram of an instruction display having an evaluation value bar, and FIG. 11D is an image diagram of an instruction display having a comment field.

FIG. 12 is an image diagram of the instruction display having an evaluation value input field for each item.

FIG. 13 is a block diagram showing a function of a storage controller.

(a) of FIG. 14 is an image diagram of an endoscopic image incidental to patient information of a name, and (b) of FIG. 14 is an image diagram of an endoscopic image in which the patient information of the name is deleted.

FIG. 15 is an explanatory diagram for describing an extraction motion picture extracted based on the result of the recognition processing.

FIG. 16 is an explanatory diagram for describing an extraction motion picture extracted based on an opinion of a doctor.

FIG. 17 is a flowchart showing a processing flow of the medical image processing device.

### DESCRIPTION OF THE PREFERRED EMBODIMENTS

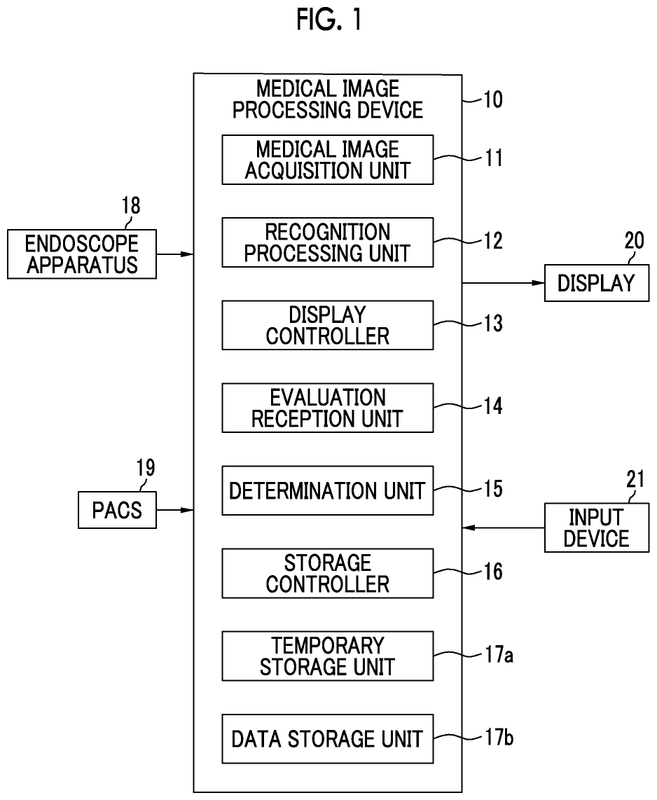

An example of a basic configuration of the present invention will be described. As shown in FIG. 1, a medical image processing device 10 comprises a medical image acquisition unit 11, a recognition processing unit 12, a display controller 13, an evaluation reception unit 14, a determination unit 15, a storage controller 16, a temporary storage unit 17a, and a data storage unit 17b. The medical image processing device 10 connects a device that can output medical image data, such as an endoscope apparatus 18, various modalities (not shown) such as X-ray examination, an examination information system (not shown) such as radiology information systems (RIS) or endoscopic information system, and a picture archiving and communication system (PACS) 19, a display device, such as a display 20, and/or an input device 21, such as a keyboard (not shown) or a touch panel of the display 20 to each other.

The medical image processing device 10 performs recognition processing of the medical image based on the medical image acquired from the endoscope apparatus 18 or the like, and performs a control of displaying, on the display 20, the medical image and a result of the recognition processing of the medical image. A user, such as a doctor, confirms the displayed medical image and the result of the recognition processing of the medical image, and uses the result of the recognition processing as diagnosis support information for diagnosis. In addition, the doctor evaluates the result of the recognition processing of the medical image based on the displayed medical image and the result of the recognition processing of the medical image. The evaluation is performed regarding whether or not the result of the recognition processing is a correct answer. The medical image processing device 10 receives the evaluation performed by the doctor, and determines, based on the evaluation, whether or not to store the medical image which is the basis of the evaluation in the data storage unit 17b. Moreover, the medical image determined to be stored is stored in the data storage unit 17b.

The medical image is, for example, a medical image handled by a PACS 19, and is mainly an examination motion picture or a still picture obtained by the examination. Specific examples thereof include an X-ray image by an X-ray examination, an MRI by an MR examination, a CT image by a CT examination, an endoscopic image by an endoscopic examination, and an ultrasound image by an ultrasound examination.

The medical image processing device 10 is operated during the examination or after the examination. Therefore, the medical image processing device 10 acquires a medical image in real time during the examination and continuously performs a series of subsequent operations, or acquires a stored medical motion picture after the examination and then continuously performs a series of subsequent operations.

The recognition processing is various pieces of processing performed using medical images, and examples thereof include detection processing of detecting a region-of-interest, such as a lesion, classification processing of classifying a disease type for the lesion, and site recognition processing of recognizing information related to a site being imaged. These pieces of processing may be combined with two or more pieces of processing, such as classifying the disease type for the lesion after detecting the region-of-interest, such as the lesion.

The recognition processing is performed by a diagnosis support learning model constructed by training the machine learning algorithm. In the diagnosis support learning model, learning and adjustment are performed such that the result of the target recognition processing is output by inputting the medical image.

The medical image and the result of the recognition processing are displayed on the display 20. The user uses the result of the recognition processing as the diagnosis support information for the diagnosis by displaying by the display 20. In addition, the result of the recognition processing is evaluated based on the display of the display 20. The evaluation includes a content indicating that the result of the recognition processing is the correct answer or is not the correct answer.

In the medical image processing device 10, it is determined whether or not to store the medical image in the data storage unit 17b based on the evaluation, and the medical image evaluated to the effect that the result of the recognition processing is not the correct answer is selected and stored in the data storage unit 17b. It should be noted that the medical image evaluated to the effect that the result of the recognition processing is the correct answer is not stored in the data storage unit 17b.

In a case of developing a function that supports the diagnosis by performing the recognition processing from the examination motion picture obtained by the examination using machine learning technique, it is important to use a large amount of examination motion picture data for learning in order to handle various clinical conditions. Therefore, it is conceivable to accumulate the examination motion pictures at each medical facility and collect the examination motion pictures for the development of the diagnosis support learning model and the like.

However, not all examination motion pictures are equally useful for training the learning model. For example, in a situation in which a developed diagnosis support learning model is already present, in a case in which the diagnosis support learning model does not misrecognize a specific examination motion picture at all, the examination motion picture means that there is little difference as information from the trained data, and the improvement of the accuracy of the diagnosis support learning model cannot be expected even in a case in which such data is added to the training data. It is not desirable to accumulate all the examination motion pictures having such a small learning merit, because it leads to the pressure on the storage which is the data storage unit 17b of the examination facility or the increase in the number of man-hours for development.

In a case in which the result of the recognition processing by the diagnosis support learning model is the correct answer, and the recognition processing is appropriately performed, there is no difference as information in the data of a target medical image from the trained data, and a possibility that the accuracy of the diagnosis support learning model is improved is low thus even in a case in which the data is added to the training data of the diagnosis support learning model. On the other hand, in a case in which the diagnosis support learning model makes a mistake in the recognition processing, that is, in a case in which the result of the recognition processing is not the correct answer, a possibility that the data of the target medical image contributes to improving the accuracy of the diagnosis support learning model is high for the opposite reason.

The medical image processing device 10 selects the medical image evaluated that the result of the recognition processing is not the correct answer, and stores the selected medical image in the data storage unit 17b. The medical image processing device 10 receives the evaluation for the result of the recognition processing from the user who confirms the result of the recognition processing, and accumulates, in the data storage unit 17b, such as the storage, the medical image data having a poor evaluation, that is, in a case in which the result of the recognition processing does not match an actual situation of a subject reflected in the medical image which is the basis of the recognition. As described above, by selecting the accumulated medical image data, it is possible to collect the medical image data that contributes to improving the accuracy of the diagnosis support learning model while suppressing the pressure on the storage or the increase in the number of man-hours for development. In particular, in a case of accumulating the examination motion pictures, the effect of reducing the storage capacity by the selection described above is great.

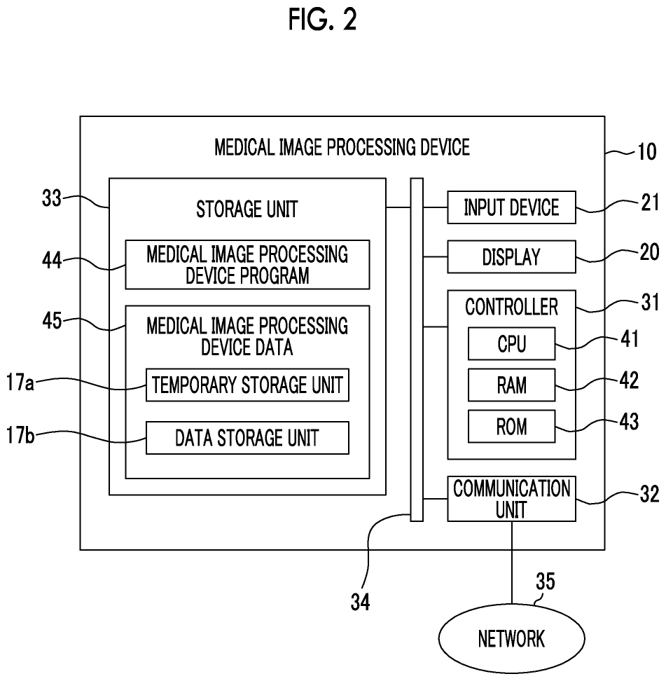

The medical image processing device 10 according to the embodiment of the present invention will be described. As shown in FIG. 2, the medical image processing device 10 according to the present embodiment is, as a hardware configuration, a computer in which the input device 21 which is an input device, the display 20 which is an output device, a controller 31, a communication unit 32, and a storage unit 33 are electrically connected to each other via a data bus 34.

The input device 21 is an input device, such as a keyboard, a mouse, or a touch panel of the display 20. The display 20 is one type of an output device. The display 20 displays various operation screens in accordance with an operation of the input device 21, such as a mouse and a keyboard. The operation screen has an operation function by a graphical user interface (GUI). The computer constituting the medical image processing device 10 can receive an input of an operation instruction from the input device 21 via the operation screens.

The controller 31 includes a central processing unit (CPU) 41, which is a processor, a random access memory (RAM) 42, and a read only memory (ROM) 43. The CPU 41 controls units of the computer in an integrated manner by loading a program stored in the storage unit 33 or the like to the RAM 42 or the ROM 43 and executing processing in accordance with the program. The communication unit 32 is a network interface that performs a transmission control of various pieces of information via a network 35. It should be noted that the RAM 42 or the ROM 43 may have the function of the storage unit 33.

The storage unit 33 is an example of a memory, and is, for example, a disk array in which a plurality of hard disk drives, solid state drives, hard disk drives, and the like built in the computer constituting the medical image processing device 10 or connected via a cable or a network are mounted. The storage unit 33 stores a control program, various application programs, various data for use in these programs, display data of various operation screens incidental to these programs, and the like.

The storage unit 33 according to the present embodiment stores various data, such as a medical image processing device program 44 and medical image processing device data 45. The medical image processing device program 44 or the medical image processing device data 45 is a program or data for performing various functions of the medical image processing device 10. The function of the medical image processing device 10 is realized by the medical image processing device program 44 and the medical image processing device data 45. In addition, the medical image processing device data 45 includes the temporary storage unit 17a and the data storage unit 17b, and data and the like temporarily stored by the medical image processing device program 44 are also stored.

The computer constituting the medical image processing device 10 may be a general-purpose server device, a personal computer (PC), or the like, in addition to a device designed exclusively for the medical image processing device 10. In addition, it is sufficient that the function of the medical image processing device 10 can be exhibited, the computer may be shared with a device performing other functions, or the function of the medical image processing device 10 can be incorporated into an endoscope management system or the like.

The medical image processing device 10 according to the present embodiment is a processor device, and the medical image processing device 10 stores a program related to the medical image processing in a storage unit 33 which is the program memory. In the medical image processing device 10, by operating the program in the program memory by the controller 31 composed of the processor, the functions of the medical image acquisition unit 11, the recognition processing unit 12, the display controller 13, the evaluation reception unit 14, the determination unit 15, and the storage controller 16 are realized (see FIG. 1).

The medical image acquisition unit 11 acquires the medical image from a device that can output the medical image. As the medical image, the examination motion picture obtained mainly by the examination is acquired. In the present embodiment, the endoscopic image obtained in the endoscopic examination using the endoscope apparatus 18 is acquired in real time. The endoscopic image is one type of the medical image, and is an image obtained by imaging the subject with an endoscope provided in the endoscope apparatus 18. In the following, a case will be described in which the endoscopic image is used as the medical image. It should be noted that the endoscopic image means the motion picture and/or the still picture. In addition, the motion picture includes individual frame images captured by the endoscope apparatus 18 in a preset number of frames.

The recognition processing unit 12 performs recognition processing on the endoscopic image acquired by the medical image acquisition unit 11. As a content of the recognition processing, in the present embodiment, the endoscopic image acquired by the medical image acquisition unit 11 is subjected to detection processing of detecting the region-of-interest, such as the lesion, in real time during the examination. In addition to the detection processing, it is possible to perform classification processing of classifying the disease type for the lesion, site recognition processing of recognizing the information related to the site being imaged, or a plurality of these pieces of processing.

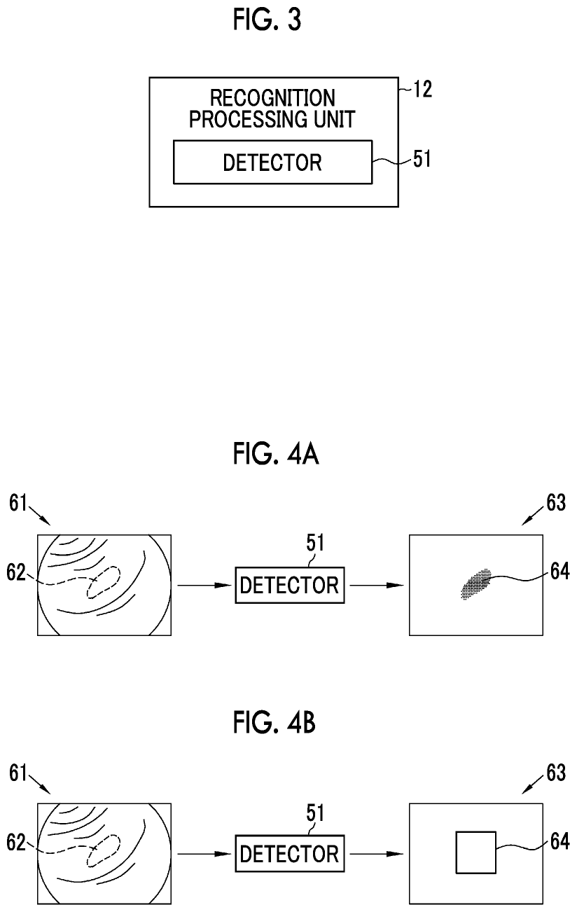

As shown in FIG. 3, the recognition processing unit 12 includes a detector 51. The detector 51 is the diagnosis support learning model that detects the region-of-interest included in the subject reflected in the endoscopic image based on the acquired endoscopic image. As shown in FIG. 4A, the detector 51 outputs a result 63 of the recognition processing in a case in which the subject reflected in an endoscopic image 61 includes a region-of-interest 62 due to the input of the endoscopic image 61. The output of the result 63 of the recognition processing displays, for example, a region itself of a detection region-of-interest 64, which is the region-of-interest 62 detected by the recognition processing, as an image form. In addition, as shown in FIG. 4B, the output of the result 63 of the recognition processing may not be the output of the region itself of the detection region-of-interest 64, but may be the output indicating the position of the detection region-of-interest 64. For example, the output of the result 63 of the recognition processing is displayed in the form of a rectangular figure indicating the detection region-of-interest 64. A notification of the result 63 of the recognition processing is given by outputting the result 63 of the recognition processing in various forms, such as an image, a figure, or a text.

The detector 51 is specifically the diagnosis support learning model constructed using a machine learning algorithm, and is a learning model that can output the presence or absence of the region-of-interest in the endoscopic image 61 as an objective variable in a case in which the endoscopic image 61 is input to the detector 51. The detector 51 is trained in advance using the machine learning algorithm using an initial image data set for the detector 51 consisting of the endoscopic image 61 and correct answer data of the region-of-interest such that the presence or absence of the region-of-interest in the endoscopic image 61 can be output as an objective variable, and the parameter is adjusted.

As the machine learning algorithm used for the detector 51, various algorithms can be used as long as the algorithms are used for supervised learning, but it is preferable to use an algorithm that outputs a good inference result as an objective variable in image recognition. For example, it is preferable to use a multi-layer neural network or a convolutional neural network, and it is preferable to use a method called so-called deep learning. In addition, in the diagnosis support learning model, techniques, such as processing on the endoscopic image 61, which is an input image, and the use of a plurality of learning models, which are commonly performed to improve the performance of the learning model, such as the improvement of the detection accuracy of the region-of-interest, and the improvement of the detection speed, may be used.

The detection result of the region-of-interest, which is the result 63 of the recognition processing, includes a position, a size or area, a shape, or the number of the regions-of-interest detected in the endoscopic image 61, and also includes a content indicating that the position or the size of the region-of-interest is 0, that is, the region-of-interest is not detected.

The display controller 13 performs the control of displaying the endoscopic image 61 and the result 63 of the recognition processing on the display 20. As a display method of the endoscopic image 61 and the result 63 of the recognition processing, it is sufficient that the doctor can confirm the endoscopic image 61 and the result 63 of the recognition processing. For example, the endoscopic image 61 can be displayed in a main region of the display 20 in which the result 63 of the recognition processing is superimposed on the endoscopic image 61, the result 63 of the recognition processing can be displayed in a sub region, or the result 63 of the recognition processing can be indicated by a text. It is possible to change to an appropriate display form in accordance with the content of the recognition processing performed by the recognition processing unit 12.

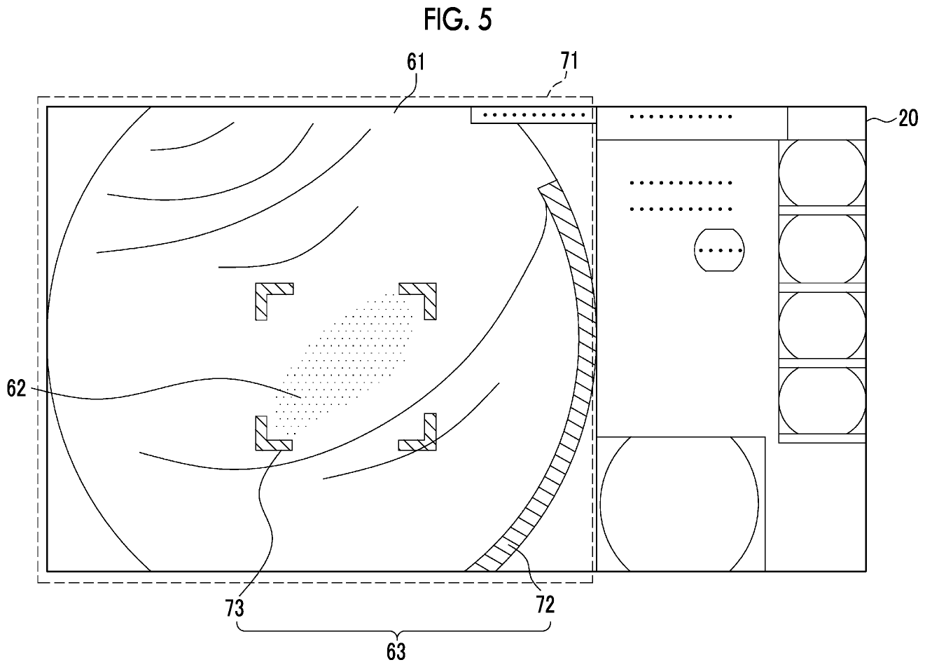

As shown in FIG. 5, in the present embodiment, since the recognition processing unit 12 performs the detection processing of detecting the region-of-interest, such as the lesion, after the examination by the endoscope apparatus 18 ends, the endoscopic image 61 and a result of the detection processing, which is the result 63 of the recognition processing, are displayed in a main region 71 of the display 20 used during the examination. In a case in which the endoscopic image 61 includes the region-of-interest 62, the doctor can confirm the region-of-interest 62 of the subject by displaying the endoscopic image 61. Moreover, the result 63 of the recognition processing can be displayed by, for example, changing a shape and color of a frame of the endoscopic image 61 close to the detected region-of-interest 62 from a normal frame, as a region-of-interest detection display frame 72. In addition, a position of the detected region-of-interest 62 can be indicated by superimposing a figure indicating the position of the detected region-of-interest 62 on the endoscopic image 61 as a region-of-interest detection display figure 73.

By looking at the endoscopic image 61, the doctor can recognize that the region-of-interest 62 is detected. The doctor can use the result 63 of the recognition processing for the diagnosis using the region-of-interest detection display frame 72 or the region-of-interest detection display figure 73 indicating the result 63 of the recognition processing. It should be noted that the examination motion picture including the endoscopic image 61 being examined and the data, such as the result 63 of the recognition processing, are stored in the temporary storage unit 17a.

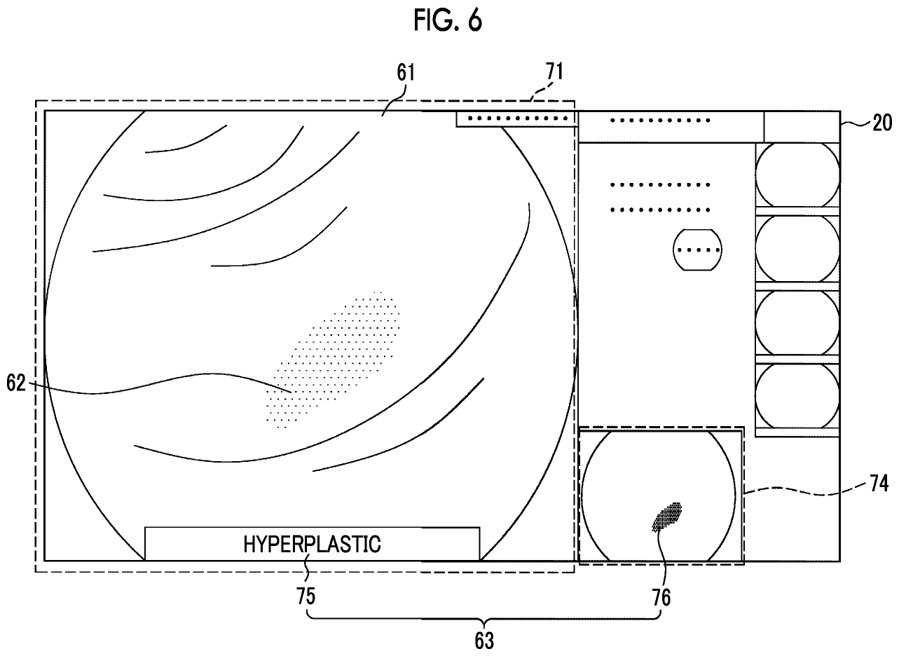

In addition, as shown in FIG. 6, for example, in a case in which the recognition processing unit 12 performs the classification processing of classifying the disease type for the lesion, after the examination by the endoscope apparatus 18 ends, in a case in which the endoscopic image 61 and a result of the classification processing, which is the result 63 of the recognition processing, are displayed on the display 20 used during the examination, the endoscopic image 61 and the result 63 of the recognition processing are displayed in the main region 71 of the display 20 used during the examination, and the result 63 of the recognition processing is also displayed in a sub region 74 of the display. As the result 63 of the recognition processing displayed in the main region 71, the result 63 of the recognition processing is displayed by a classification result display text 75. The result 63 of the recognition processing is displayed by a text, such as “HYPERPLASTIC”. In addition, in the sub region 74 as well, as the result 63 of the recognition processing, the position of the region-of-interest and the disease type of the region-of-interest are displayed by color by a classification result color display 76. In FIG. 6, the classification result color display 76 is displayed in color indicating that the region-of-interest is hyperplasia.

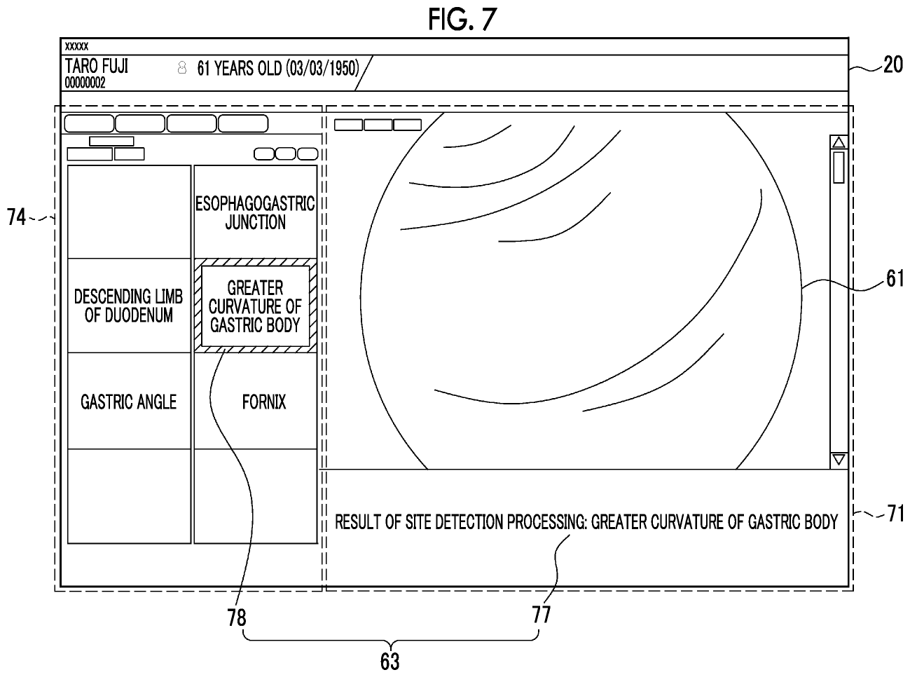

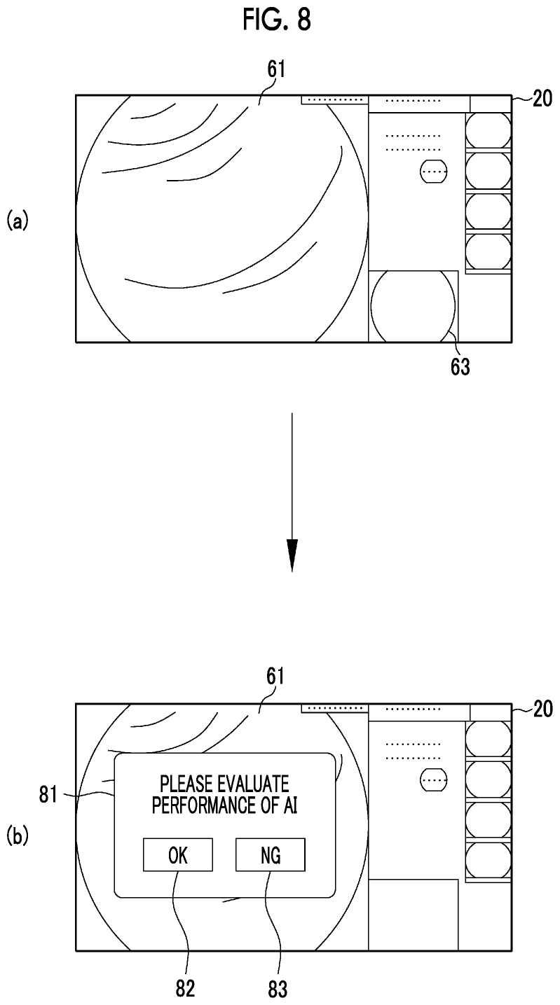

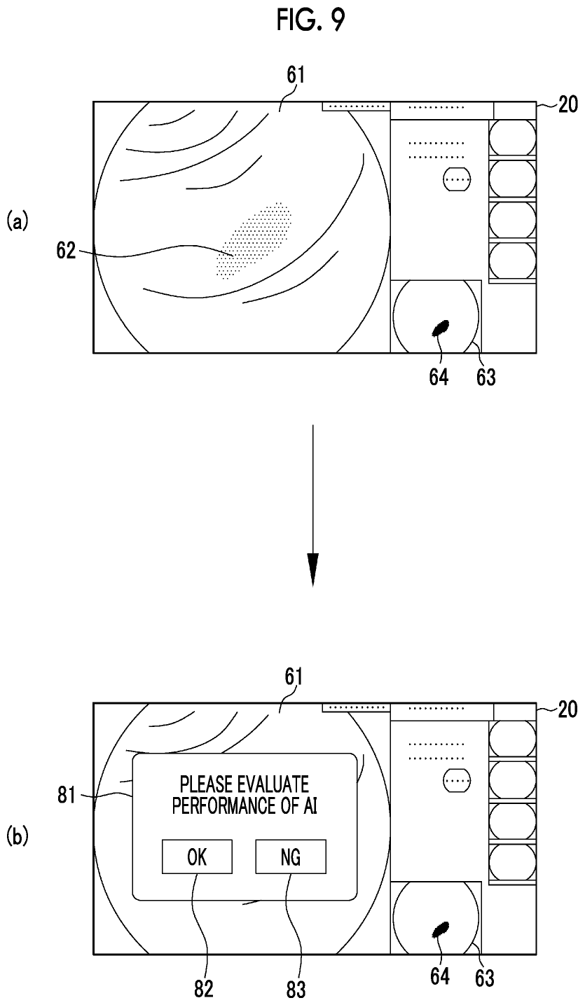

In addition, as shown in FIG. 7, for example, in a case in which the recognition processing unit 12 performs site recognition processing of recognizing information related to the site, after the examination by the endoscope apparatus 18 ends, in a case in which the endoscopic image 61 and a result of the site recognition processing, which is the result 63 of the recognition processing, are displayed in the main region 71 of the display 20 for creating the examination report, for example, the endoscopic image 61 and a site name display text 77 are displayed in the main region 71 of the display 20 for creating the examination report, and the result 63 of the recognition processing is also displayed in the sub region 74 of the display by highlighting 78 a tile of the site name.

The evaluation reception unit 14 receives an evaluation related to the result 63 of the recognition processing from the doctor based on the endoscopic image 61 and the result 63 of the recognition processing of the endoscopic image 61, which are displayed. The evaluation need only be an evaluation related to the result 63 of the recognition processing, and can be, for example, a viewpoint of whether or not the result 63 of the recognition processing is the correct answer, or a viewpoint of whether the performance or accuracy of the detector 51 is high or low. It should be noted that the fact that the result 63 of the recognition processing is the correct answer means that the result 63 of the recognition processing matches the actual situation of the subject reflected in the medical image which is the basis of the recognition processing. In addition, it can be said that the high performance or accuracy of the detector 51 means that the degree of matching of the result 63 of the recognition processing with the actual situation of the subject reflected in the medical image which is the basis of the recognition processing.

A format of the evaluation can be determined depending on a case, for example, selection from two options, selection from three or more options, inputting a numerical value as an evaluation value, and adding a comment in a free description format, or a format, such as a combination thereof, can be adopted. In the present embodiment, the format of selection from two options related to a viewpoint of a quality of the performance of the detector 51, such as whether the performance of the detector 51 is high or low, is adopted.

A timing of the evaluation may be performed during the examination using the endoscope apparatus 18, or may be after the examination ends. For example, in a case of performing the evaluation during the examination, for example, the evaluation is performed by assigning an option of the evaluation to a scope button provided in the endoscope. Among the options of the evaluation, it is preferable to assign the evaluation that the performance of the detector 51 is low to one of the scope buttons. Specifically, the doctor confirms the display 20 (see FIG. 6) in which the endoscopic image 61 is displayed in the main region 71 and the result 63 of the recognition processing is displayed in the sub region 74, and presses the scope button in a case in which the result 63 of the recognition processing displayed in the sub region 74 does not match the endoscopic image 61 displayed in the main region 71, thereby completing the evaluation of the result 63 of the recognition processing displayed in the sub region 74.

In a case in which the evaluation is performed after the examination ends, it is preferable that the evaluation reception unit 14 receive the evaluation after the endoscopic image 61 and the result 63 of the recognition processing of the endoscopic image are displayed on the display 20 after the examination ends. In a case in which the evaluation is performed after the examination ends, the endoscopic image 61 and the like may be displayed on the display 20 displaying the endoscopic image 61 and the like during the examination, or the endoscopic image 61 and the like may be displayed on the display 20 different from the display 20 displaying the endoscopic image 61 during the examination. For example, in a case in which the evaluation is performed after the examination ends, the endoscopic image 61 and the like may be displayed on the display 20 of a terminal for creating the examination report after the examination.

The evaluation can be performed, for example, by the doctor selecting the options displayed on the display 20 by the display controller 13. The doctor completes the evaluation by selecting the displayed options of the evaluation. In addition, the display controller 13 may display, on the display 20, an instruction display prompting the doctor to evaluate the performance of the detector 51. In this case, the evaluation reception unit 14 receives the evaluation after the instruction display is displayed on the display 20.

It should be noted that, during the examination or after the examination ends, the display controller 13 may use the display 20 displaying the endoscopic image 61, the display 20 displaying the instruction display, the display 20 displaying the result of the recognition processing of the endoscopic image 61 as the displays 20 different from each other, may use two of the displays 20 as the same display 20, or may use all three displays 20 as the same display 20. The display 20 can be the display 20 used in the endoscopic examination, the display 20 used to create the examination report after the examination ends, the display 20 of a small terminal, such as a tablet, or the like.

In the present embodiment, after the examination ends, the endoscopic image 61 and the like are displayed on the display 20 displaying the endoscopic image 61 during the examination, and the evaluation is performed. As shown in (a) of FIG. 8, specifically, in the display 20 displaying the endoscopic image 61 and the like during the examination, as shown in (b) of FIG. 8, after the examination ends after a lapse of time, the display controller 13 performs a control of displaying the endoscopic image 61 and the like, and an instruction display 81 including an OK button 82 and an NG button 83. As the instruction display 81, “please evaluate the performance of AI” is displayed. Here, artificial intelligence (AI) indicates the recognition processing.

Based on the endoscopic image 61 confirmed during the examination or after the examination ends, and the result 63 of the recognition processing of the endoscopic image 61, the doctor selects the OK button 82 on the touch panel of the display 20 in a case in which the performance of the recognition processing by the detector 51 is evaluated as high, and selects the NG button 83 on the touch panel of the display 20 in a case in which the performance is evaluated as low, thereby completing the evaluation.

It should be noted that, as shown in (a) of FIG. 9, the endoscopic image 61 and the result 63 of the recognition processing may be shown during the examination, and as shown in (b) of FIG. 9, the result 63 of the recognition processing may be shown also after the examination ends after a lapse of time. In addition, only a part of the result 63 of the recognition processing may be shown. As a result, the doctor can be reminded of the performance of the recognition processing during the examination, and the doctor can perform a more accurate evaluation more easily.

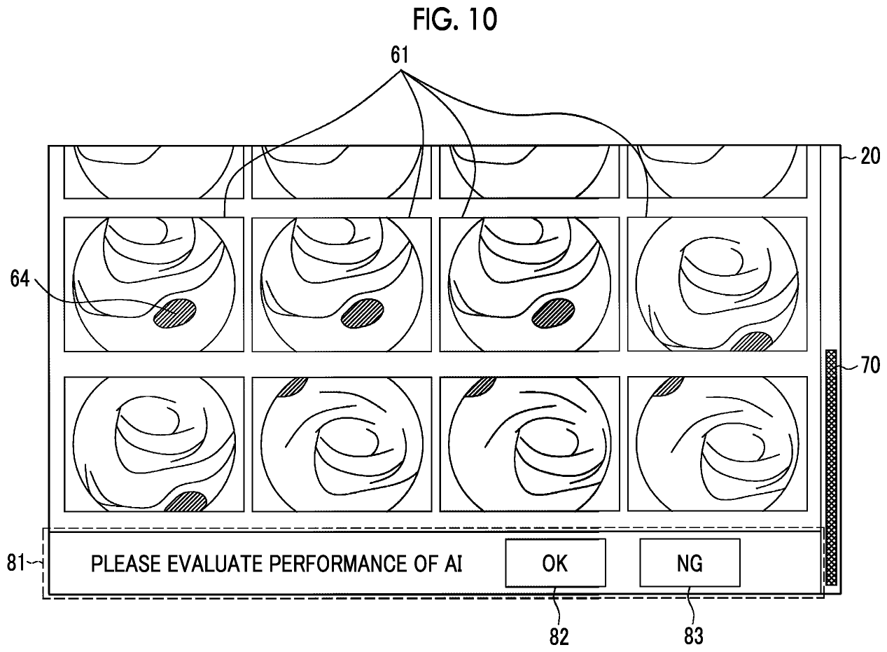

In addition, as shown in FIG. 10, after the examination ends, a plurality of endoscopic images 61 and each result 63 of the recognition processing may be displayed in a list on the display 20 as thumbnail images. By moving with a scroll bar 70, the entire list of the plurality of endoscopic images 61 can be viewed. It should be noted that, depending on the figure, a reference numeral may be added only to a part of the images in order to prevent complication.

The plurality of endoscopic images 61 may be a plurality of frame images of the endoscopic images 61 selected in accordance with a preset condition. As a condition, for example, in a case in which the recognition processing is the detection processing, the frame image in which the region-of-interest or the like is detected is used, in a case in which the recognition processing is the classification processing for the lesion and the like, the frame image in which the classification result is output is used, and in a case in which the recognition processing is the site recognition processing, the frame image in which a specific site result of the recognition is output is used. Moreover, these frame images are displayed in a list in an order of time points at which the frame images are acquired, or in an order of importance of the results of the recognition processing set in advance.

It should be noted that it is preferable that the frame image to be displayed in a list also shows the result 63 of the recognition processing. In FIG. 10, the result 63 of the recognition processing is the result of the detection processing, and is shown by superimposing the detection region-of-interest 64 on the endoscopic image 61. By displaying the list of the plurality of endoscopic images 61 on the display 20, the doctor can grasp the performance of the recognition processing in the entire examination in a short time, so that more accurate evaluation can be performed in a short time.

In addition, the evaluation can be performed by a numerical value, such as the evaluation value. In this case, the evaluation is performed by, for example, the evaluation value indicating a degree to which the result 63 of the recognition processing is the correct answer. The determination unit 15 determines to store the endoscopic image 61 in the data storage unit 17b in a case in which the evaluation value is within a preset range indicating that the degree to which the result 63 of the recognition processing is the correct answer is low.

As the evaluation value, it is possible to use a percentage of 100 in a case in which the degree to which the result 63 of the recognition processing is the correct answer is the highest, and a percentage of 1 in which case in which the degree to which the result 63 of the recognition processing is the correct answer is the lowest. The evaluation value may be input by a numerical value, or may be selected from several stages. For example, the evaluation value may be selected from five stages, such as 1, 30, 60, 90, and 100.

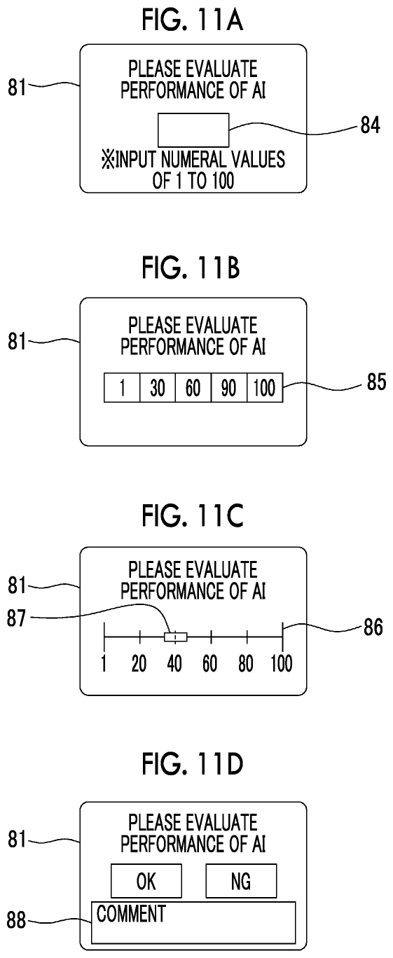

Specifically, the instruction display 81 in a case in which the evaluation value is used can be as follows. As shown in FIG. 11A, for example, the instruction display 81 may include an evaluation value input frame 84. In the evaluation value input frame 84, the evaluation value is input as a numerical value of 1 to 100. In addition, as shown in FIG. 11B, the instruction display 81 may include an evaluation value input button 85. The evaluation value input button 85 includes, for example, buttons of five stages, such as 1, 30, 60, 90, and 100. In addition, as shown in FIG. 11C, an evaluation value bar 86 and a slider 87 may be included. The slider 87 is moved on a desired numerical value on the evaluation value bar 86 to designate the evaluation value. In addition, as shown in FIG. 11D, the instruction display 81 may include a comment field 88 in addition to the OK button 82 and the NG button 83. A comment can be described in the comment field 88 by a free description method. The content described in the comment field 88 may be used for the evaluation, or can be used for the training data as the annotation for the endoscopic image 61 which is the target of the evaluation.

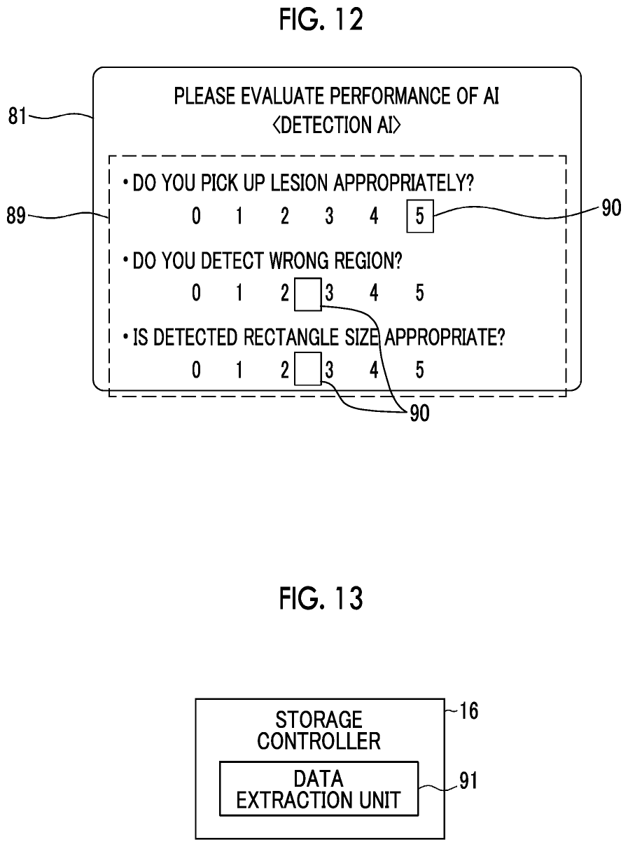

As shown in FIG. 12, the instruction display 81 may include an evaluation value input field 89 to which the evaluation value for each item is input. In the evaluation value input field, the evaluation value may be selected by a button, a figure, or the like, or a numerical value may be input. For example, in a case in which, as the recognition processing, the detection processing of detecting the region-of-interest is performed, a specific item, such as “Do you pick up the lesion appropriately?”, “Do you detect the wrong region?”, or “Is the detected rectangle size appropriate?”, is displayed. The doctor selects one of six stages from 0 to 5 as the evaluation value by moving the selection frame 90, which is a rectangular frame, on the screen. Moreover, a comprehensive evaluation value may be calculated from the evaluation values of these plurality of items, and it may be determined whether or not to store the endoscopic image 61 based on the comprehensive evaluation value.

In addition, it is preferable that the evaluation result, such as the evaluation value for each item, be associated with the endoscopic image 61. Since the evaluation results, such as these evaluation values, are the evaluation values associated with the endoscopic image 61, the evaluation results can be used as a guideline for the development policy of the detector 51 and the like after accumulating and collecting the endoscopic image 61. In addition, the evaluation results can be used as useful data that can be used in various ways by performing statistical processing for each item.

Based on the evaluation, the determination unit 15 determines whether or not to store the endoscopic image 61, which is the basis of the evaluation, in the data storage unit 17b. The endoscopic image 61, which is the basis of the evaluation, is the endoscopic image 61 that is the target of the recognition processing. In a case in which the endoscopic image 61 is the examination motion picture, the entire examination motion pictures may be stored, or a part of the examination motion picture may be selected and stored. In a case in which the endoscopic image 61 is the still picture, one still picture which is the target of the recognition processing may be used, or a plurality of still pictures including this still picture may be used.

In the determination, it is determined whether or not the evaluation related to the result of the recognition processing is low. Specifically, the determination is made in accordance with an evaluation format. For example, as in the present embodiment, in a case in which the options of the evaluation are two options, that is, the performance of the recognition processing is high or low, it is determined to store the endoscopic image 61 evaluated by the option of low among the two options in the data storage unit 17b. In a case in which there are three or more options of the evaluation, storing the endoscopic image 61, which is the basis of the evaluation, in the data storage unit 17b is set in advance in a case in which any of three or more options is selected. In addition, in a case in which the evaluation is performed by the evaluation value, a threshold value is set in advance, and the endoscopic image 61 is stored in the data storage unit 17b in a case in which the evaluation value is equal to or less than the threshold value.

The storage controller 16 performs a control of storing, in the data storage unit 17b, the endoscopic image 61 determined to be stored. Regarding the endoscopic image 61 determined to be stored, in a case in which the motion picture and/or the still picture during the examination and after the examination is stored in the temporary storage unit 17a, the endoscopic image 61 stored in the temporary storage unit 17a is stored in the data storage unit 17b. Therefore, the endoscopic image 61 is stored in the data storage unit 17b as the still picture and/or as the motion picture.

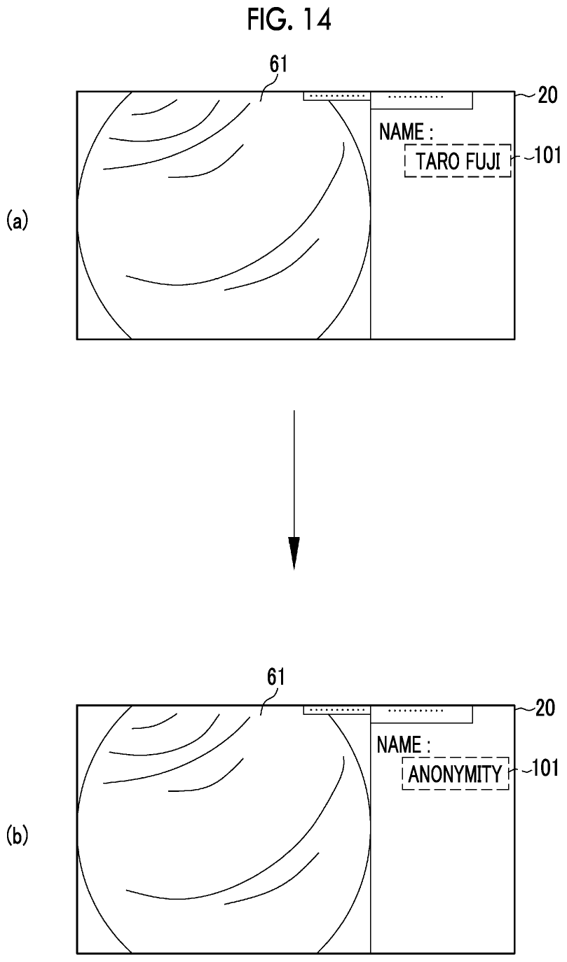

In addition, as shown in FIG. 13, the storage controller 16 comprises a data extraction unit 91, and the data extraction unit 91 may extract a part of the endoscopic images 61 from the endoscopic images 61 stored in the temporary storage unit 17a. The data storage unit 17b stores the endoscopic image 61 extracted by the data extraction unit 91 in the data storage unit 17b. Details of the data extraction unit 91 will be described below.

As in the present embodiment, in a case in which the options of the evaluation are two options, that is, the performance of the recognition processing is high or low, the storage controller 16 moves the motion picture being examined, which is stored in the temporary storage unit 17a after the examination, to the data storage unit 17b in a case in which the NG button 83 is selected, and deletes the motion picture being examined, from the temporary storage unit 17a in a case in which the OK button 82 is selected. As described above, the data storage unit 17b accumulates the examination motion pictures evaluated by the doctor as having low recognition processing performance. Moreover, both the temporary storage unit 17a and the data storage unit 17b can save storage.

It should be noted that the temporary storage unit 17a or the data storage unit 17b may be provided inside the medical image processing device 10 or may be provided outside the medical image processing device 10. In addition, the data storage unit 17b may accumulate the evaluation by the doctor, details of the evaluation, and the like incidentally to the endoscopic image 61. As a result, in addition to the reduction of the storage capacity, the detailed evaluation by the user for the recognition processing can also be reflected in the development of the detector 51 and the like.

As described above, the medical image processing device 10 can selectively accumulate the endoscopic image 61 useful for learning. The endoscopic image 61, which is not considered to be useful for learning, is not accumulated, and thus the capacity of the data storage unit 17b can be greatly saved. Moreover, since the selection can be performed by one operation, such as one click, it does not take time and effort. Since the selected endoscopic image 61 is the endoscopic image 61 for which the recognition processing unit 12 makes a mistake in the recognition processing, it is an endoscopic image particularly useful for training the diagnosis support learning model, such as a detector 51, in the recognition processing unit 12, and is also the endoscopic image 61 useful for training the diagnosis support learning model other than the detector 51. Since the endoscopic image 61 useful for learning is automatically accumulated in one storage, it is easy to be moved or managed.

Then, storing the endoscopic image 61 in the data storage unit 17b will be further described. First, the data stored in the temporary storage unit 17a during the examination or after the examination ends may be data in which various pieces of information are associated with the endoscopic image 61. Examples of the information associated with the endoscopic image 61 include the result of the recognition processing by the recognition processing unit 12, and the diagnosis information related to the diagnosis made by the doctor during the examination or after the examination ends. In a case in which the endoscopic image 61 with which these information are associated is stored in the data storage unit 17b, the data storage unit 17b also receives the diagnosis information related to the diagnosis made by the doctor on the subject, and the like. The data storage unit 17b stores the endoscopic image 61 and the diagnosis information in the data storage unit 17b in association with each other.

In a case in which the result of the recognition processing is added, it is preferable to store the result of the recognition processing together with the information of the time point at which the target endoscopic image 61 is acquired or the time point at which the result of the recognition processing is obtained. This makes it easy to specify the time region in a case in which the result of the recognition by the recognition processing unit 12 is wrong. In addition, the diagnosis information related to the diagnosis made by the doctor may be information, such as an opinion or a diagnosis added in a case in which the doctor creates the examination report after the examination ends. As a result, by the data of the endoscopic image 61 with which the information related to the first opinion or the diagnosis of the doctor stored in the data storage unit 17b is associated, it is possible to collect the endoscopic image 61 to which a correct answer label or the annotation is added in a case of being used for training the detector 51 and the like.

On the other hand, in a case in which individual information is associated with the endoscopic image 61, it is preferable to anonymize the information for specifying an individual patient associated with the accumulated endoscopic image 61. For example, in a case in which the patient information incidental to the endoscopic image 61 includes individual information related to an individual having the subject, it is preferable to delete the individual information from the endoscopic image 61 and store the endoscopic image 61 in the data storage unit 17b.

In the examination using the endoscope apparatus 18, the endoscopic image 61 may be incidental to the patient information for specifying the patient. For example, the medical images or examination information data including the motion pictures are unified by a standard of digital imaging and communications in medicine (DICOM), and this standard includes the individual information of the patient, such as the patient name.

As shown in (a) of FIG. 14, in a case in which the endoscopic image 61 is incidental to a name as the patient information, the name is displayed in a name display field 101. In a case in which the endoscopic image 61 incidental to the name as the patient information is stored in the data storage unit 17b, the data storage unit 17b deletes the individual information, such as the name that is not necessary as the training data. As shown in (b) of FIG. 14, after the name is deleted, “anonymity” or the like may be displayed as the name displayed in the name display field 101 such that the name can be understood that the name is deleted.

From the viewpoint of the individual information protection, it is not permitted to take the data of each facility out of the facility in a case in which the information for specifying the individual is included. Therefore, by performing anonymization processing at a time point of accumulation in the data storage unit 17b, the problem described above during the collection of the data, such as the endoscopic image 61, can be avoided. It should be noted that, unless the patient is specified, useful information as the training data can be selected and left. For example, the patient name is deleted, but the age, medical history, disease name, and the like may not be deleted in some cases because the age, medical history, disease name, and the like may be useful as the training data.

In a case of storing, in the data storage unit 17b, the endoscopic image 61 stored in the temporary storage unit 17a and various pieces of information incidental to and associated with the endoscopic image 61, the storage controller 16 may accumulate the endoscopic image 61 and all of various pieces of information, or may extract and accumulate a part of the endoscopic images 61 and various pieces of information by the data extraction unit 91. For the endoscopic image 61 determined to be stored, in a case in which the storage controller 16 extracts and accumulates a part of the endoscopic images 61, it is preferable to perform a control of deleting a portion of the endoscopic image 61 that is not stored in the data storage unit 17b from the temporary storage unit 17a or the data storage unit 17b. As a result, it is possible to further reduce the storage capacity and the number of man-hours for development while leaving the endoscopic image 61 and various pieces of information incidental to the endoscopic image 61 useful for training the detector 51.

For example, in a case in which the result 63 of the recognition processing of the endoscopic image 61 includes a specific content set in advance, the data extraction unit 91 may store, in the data storage unit 17b, the endoscopic image 61 acquired in a preset period including the time point at which the endoscopic image 61, which is a target of the recognition processing of the endoscopic image 61, is acquired. Therefore, the data extraction unit 91 extracts the data after grasping the endoscopic image 61 and various pieces of information associated with the endoscopic image 61.

Examples of the preset specific content include a content indicating that the region-of-interest 62 is detected by the result 63 of the recognition processing, in a case in which the recognition processing unit 12 performs the detection processing of detecting the region-of-interest 62. In this case, only the motion picture for a preset period including the time point at which the endoscopic image 61, which is the target of the recognition processing in a case in which the result 63 of the recognition processing is the result of detecting the region-of-interest 62, is acquired, is extracted, and the endoscopic image 61 is stored in the data storage unit 17b.

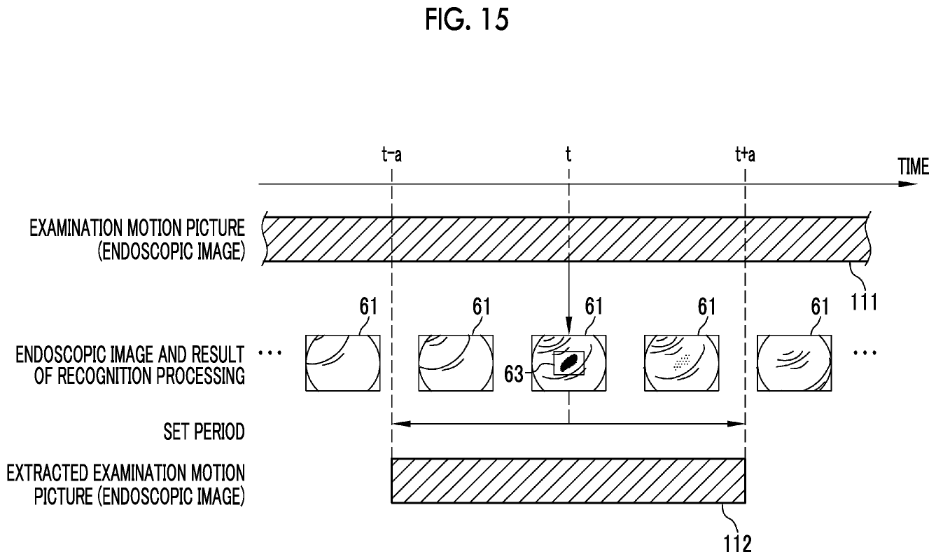

As shown in FIG. 15, specifically, in a case in which there is the endoscopic image 61 in which the region-of-interest 62 is detected as the result 63 of the recognition processing, in the endoscopic image 61 which is an examination motion picture 111, the data extraction unit 91 extracts the examination motion picture in a preset period including a time point t at which the endoscopic image 61 is acquired, and uses the extracted examination motion picture as an extraction motion picture 112. Moreover, the extraction motion picture 112 is stored in the data storage unit 17b. Here, since the preset period is a period a before and after the time point t at which the endoscopic image 61 in which the region-of-interest 62 is detected is acquired, the extraction motion picture 112 is the extraction motion picture 112 obtained by extracting the period from a time point t−a to a time point t+a in the examination motion picture 111.

In addition, in a case in which the doctor adds a specific opinion which is the diagnosis information to the endoscopic image 61, the endoscopic image 61 may be extracted in accordance with the content of the diagnosis information, and then the extracted endoscopic image 61 may be stored in the data storage unit 17b. The specific opinion can be appropriately set, such as finding the region-of-interest 62, finding a specific lesion, or finding a remitted region. Therefore, the data extraction unit 91 extracts the data after grasping the endoscopic image 61 and the opinion of the doctor associated with the endoscopic image 61.

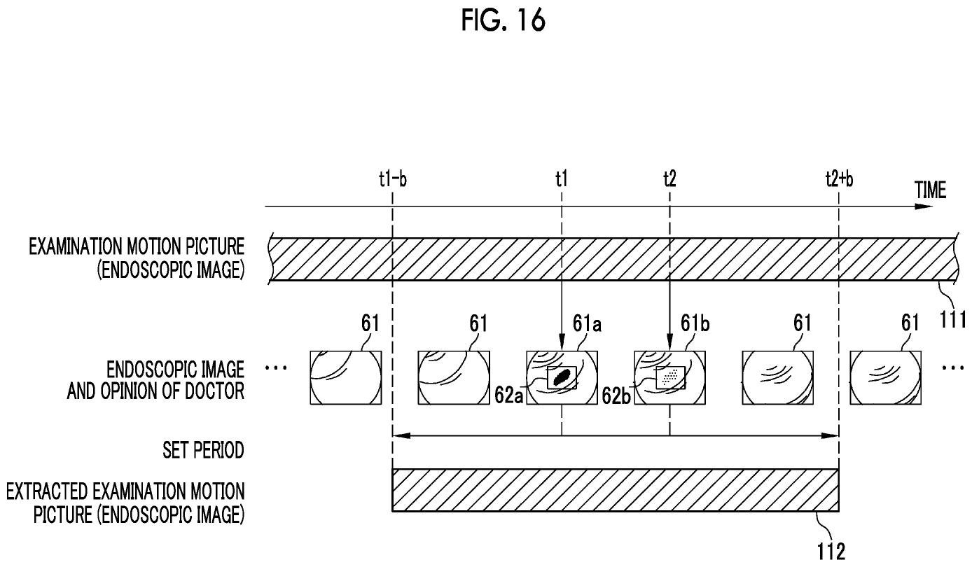

As shown in FIG. 16, specifically, in a case in which, for an endoscopic image 61a and an endoscopic image 61b, which are the examination motion pictures 111, the doctor designates a region-of-interest 62a for the endoscopic image 61a and designates a region-of-interest 62b for the endoscopic image 61b as the opinion, based on the these pieces of diagnosis information of the doctor added to the endoscopic image 61a and the endoscopic image 61b, the data extraction unit 91 may extract the motion picture of a preset period including a time point t1 at which the endoscopic image 61a in which the region is designated by the doctor is acquired, and a time point t2 at which the endoscopic image 61b in which the region is designated by the doctor is acquired, and may store the endoscopic image 61 in the data storage unit 17b. In a case in which there are the plurality of endoscopic images 61 extracted in accordance with the content of the diagnosis information, it is preferable to extract the examination motion picture 111 such that these endoscopic images 61 are included.

Here, since the preset period is a period b before and after the time point t at which the endoscopic image 61 in which the region-of-interest is detected is acquired, the extraction motion picture 112 can be from a set period before the acquisition time point of the endoscopic image 61 acquired at the oldest time point to a set period after the acquisition time point of the endoscopic image 61 acquired at the latest time point, among the plurality of endoscopic images 61 included in the extraction motion picture 112. Therefore, in the case of FIG. 16, the extraction motion picture 112 is obtained by extracting the period from a time point t1−b to a time point t2+b in the examination motion picture 111.

The data extraction unit 91 generates the extraction motion picture 112 extracted from the examination motion picture 111 under a specific condition, so that the storage controller 16 performs the control of storing the extraction motion picture 112 in the data storage unit 17b instead of the entire examination motion picture 111. Therefore, it is possible to select a scene useful for learning from the entire examination motion picture 111 in accordance with the setting made in advance and store the selected scene in the data storage unit 17b. As a result, it is possible to greatly suppress the pressure on the storage. In addition, since the extraction motion picture 112 is associated with information, such as the result 63 of the recognition processing or the specific opinion of the doctor, it can be effectively used as the training data to which the annotation is added.

Then, the endoscopic image 61 that is not determined to be stored by the storage controller 16 will be described. The storage controller 16 may perform a control of deleting the endoscopic image 61 that is not determined to be stored by the storage controller 16, from the temporary storage unit 17a. As a result, it is possible to save the storage of the temporary storage unit 17a in addition to the data storage unit 17b.

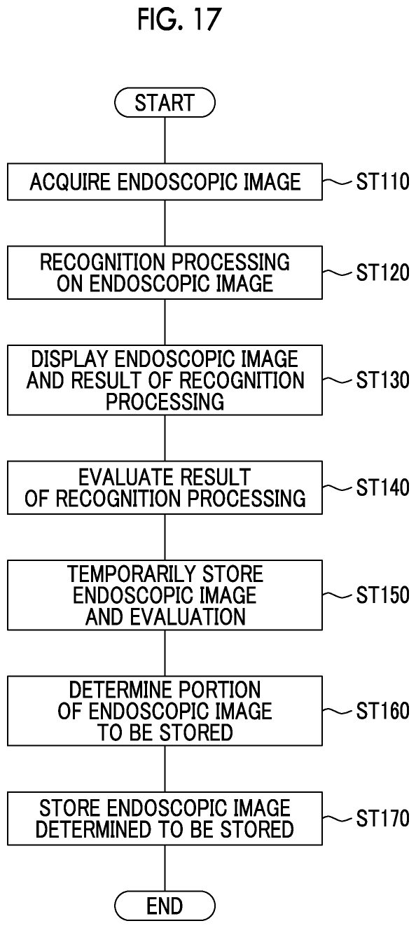

Then, a flow of processing by the medical image processing device 10 according to the present embodiment will be described. As shown in FIG. 17, the medical image acquisition unit 11 acquires the endoscopic image 61 obtained by the endoscope apparatus 18 (step ST110). The subject is reflected in the endoscopic image 61. The recognition processing unit 12 performs the recognition processing on the endoscopic image 61 acquired by the medical image acquisition unit 11 (step ST120).

The doctor looks at the result 63 of the recognition processing and the endoscopic image 61, which are displayed on the display 20, and proceeds with the examination. After the examination ends, the display controller 13 performs the control of displaying, on the display 20, the endoscopic image 61 acquired by the medical image acquisition unit 11, and also performs the control of displaying, on the display 20, the result 63 of the recognition processing (step ST130). The doctor looks at the endoscopic image 61 and the result 63 of the recognition processing of the endoscopic image 61, which are displayed on the display 20, and evaluates the result 63 of the recognition processing (step ST140). The evaluation is the evaluation related to the quality of the recognition processing. The evaluation is stored in the temporary storage unit 17a in association with the endoscopic image 61 (step ST150).

The determination unit 15 determines the endoscopic image 61 stored in the temporary storage unit 17a and the content of the evaluation associated therewith, and determines to store the endoscopic image 61 having the evaluation equal to or less than a preset level, in the data storage unit 17b. The storage controller 16 determines whether to store all the endoscopic images 61 determined to be stored in the data storage unit 17b or to extract and store a part of the endoscopic images 61 by the determination unit 15, and determines a storage portion of the endoscopic image 61 (step ST160). The storage controller 16 performs the control of storing all or a part of the endoscopic images 61 determined to be stored by the storage controller 16 in the data storage unit 17b (step ST170).

The embodiment described above includes the program for medical image processing causing the computer to execute a process of acquiring the medical image in which the subject is reflected, a process of performing the recognition processing on the medical image, a process of performing the control of displaying the medical image and the result of the recognition processing of the medical image on the display, a process of receiving the evaluation related to the result of the recognition processing from the user based on the displayed medical image and the displayed result of the recognition processing of the medical image, a process of determining whether or not to store the medical image, which is a target of the evaluation, in the data storage unit based on the evaluation, and a process of performing the control of storing, in the data storage unit, the medical image determined to be stored.

In the embodiment described above, a hardware structure of a processing unit, such as the medical image acquisition unit 11, the recognition processing unit 12, the display controller 13, the evaluation reception unit 14, the determination unit 15, and the storage controller 16 included in the medical image processing device 10, which is the processor device, is various processors as described below. Examples of the various processors include a central processing unit (CPU), which is a general-purpose processor that executes software (program) to function as various processing units, a programmable logic device (PLD), which is a processor of which a circuit configuration is changeable after manufacturing, such as a field programmable gate array (FPGA), and a dedicated electric circuit, which is a processor having a circuit configuration designed exclusively for executing various pieces of processing.

One processing unit may be composed of one of these various processors, or may be composed of a combination of two or more same type or different type of processors (for example, a plurality of FPGAs, or a combination of a CPU and an FPGA). In addition, a plurality of processing units may be composed of one processor. As an example in which the plurality of processing units are composed of one processor, first, there is a form in which one processor is composed of a combination of one or more CPUs and software, and this processor functions as the plurality of processing units, as represented by a computer, such as a client or a server. Second, as represented by a system on chip (SoC) or the like, there is a form of using a processor that realizes the functions of the entire system including the plurality of processing units with one integrated circuit (IC) chip. As described above, various processing units are composed of one or more of the various processors described above as the hardware structure.

More specifically, the hardware structure of these various processors is an electrical circuit (circuitry) in a form of a combination of circuit elements, such as semiconductor elements.

### EXPLANATION OF REFERENCES

10: medical image processing device

11: medical image acquisition unit

12: recognition processing unit

13: display controller

14: evaluation reception unit

15: determination unit

16: storage controller

17a: temporary storage unit

17b: data storage unit

18: endoscope apparatus

19: PACS

20: display

21: input device

31: controller

32: communication unit

33: storage unit

34: data bus

35: network

41: CPU

42: RAM

43: ROM

44: medical image processing device program

45: medical image processing device data

51: detector

61, 61a, 61b: endoscopic image

62, 62a, 62b: region-of-interest

63: result of recognition processing

64: detection region-of-interest

70: scroll bar

71: main region

72: region-of-interest detection display frame

73: region-of-interest detection display figure

74: sub region

75: classification result display text

76: classification result color display

77: site name display text

78: highlighting tile of site name

81: instruction display

82: OK button

83: NG button

84: evaluation value input frame

85: evaluation value input button

86: evaluation value bar

87: slider

88: comment field

89: evaluation value input field

90: selection frame

91: data extraction unit

101: name display field

111: examination motion picture

112: extraction motion picture

ST110 to ST170: step

What is claimed is:

1. A medical image processing device comprising:
a processor configured to:
acquire a medical image in which a subject is reflected;
perform recognition processing on the medical image;
display, on a display, the medical image and a result of the recognition processing of the medical image;
receive an evaluation related to the result of the recognition processing from a user;
determine whether or not to store the medical image, which is a target of the evaluation, in a data storage based on the evaluation; and
store, in the data storage, the medical image determined to be stored.

2. The medical image processing device according to claim 1,
wherein the processor is configured to:
display, on the display, an instruction display prompting the user for the evaluation; and
receive the evaluation after the instruction display is displayed.

3. The medical image processing device according to claim 1,
wherein the processor is configured to:
acquire the medical image during examination; and
display the instruction display at a preset timing after the end of the examination.

4. The medical image processing device according to claim 1,
wherein the processor is configured to:
acquire the medical image during examination;
display, on the display, the medical image and the result of the recognition processing of the medical image at a preset timing after the examination ends; and
receive the evaluation after the medical image and the result of the recognition processing of the medical image are displayed on the display.

5. The medical image processing device according to claim 3,
wherein the processor is configured to differentiate the display on which the medical image and the result of the recognition processing of the medical image is displayed during the examination, and the display on which the medical image and the result of the recognition processing of the medical image is displayed after the end of the examination.

6. The medical image processing device according to claim 1,
wherein the evaluation is performed based on an evaluation value indicating a degree to which the result of the recognition processing is correct, and
the processor is configured to determine to store the medical image in the data storage in a case in which the evaluation value is within a preset range indicating that the degree to which the result of the recognition processing is correct is low.

7. The medical image processing device according to claim 1,
wherein the processor is configured to temporarily store the medical image in a temporary storage.

8. The medical image processing device according to claim 7,
wherein the processor is configured to, in a case in which it is determined to store the medical image, store the medical image stored in the temporary storage in the data storage.

9. The medical image processing device according to claim 7,
wherein the processor is configured to, in a case in which it is determined to store the medical image, extract a part of the medical image from the medical image stored in the temporary storage and then store the extracted part of the medical image in the data storage.

10. The medical image processing device according to claim 1,
wherein the processor is configured to store in the data storage, in a case in which the result of the recognition processing of the medical image includes a preset specific content, the medical image acquired in a preset period including a time point at which the medical image, which is a target of the recognition processing of the medical image, is acquired.

11. The medical image processing device according to claim 1,
wherein the processor is configured to:
receive diagnosis information related to a diagnosis made by the user on the subject; and
store, in the data storage, the medical image and the diagnosis information in association with each other.

12. The medical image processing device according to claim 11,
wherein the processor is configured to extract the medical image in accordance with a content of the diagnosis information, and then store the extracted medical image in the data storage.

13. The medical image processing device according to claim 1,
wherein the processor is configured to, in a case in which the medical image includes individual information related to an individual having the subject, delete the individual information from the medical image, and then store the medical image in the data storage.

14. The medical image processing device according to claim 1,
wherein the processor is configured to store, in the data storage, the medical image and the result of the recognition processing of the medical image in association with each other.

15. The medical image processing device according to claim 1,
wherein the processor is configured to store, in the data storage, the medical image as a still picture and/or a motion picture including the medical image.

16. The medical image processing device according to claim 2,
wherein the processor is configured to differentiate the display on which the instruction display is displayed, the display on which the medical image is displayed, and the display on which the result of the recognition processing of the medical image is displayed, or make at least two of them the same.

17. An operation method of a medical image processing device, the method comprising:
acquiring a medical image in which a subject is reflected;
performing recognition processing on the medical image;
displaying, on a display, the medical image and a result of the recognition processing of the medical image;
receiving an evaluation related to the result of the recognition processing from a user;
determining whether or not to store the medical image, which is a target of the evaluation, in a data storage based on the evaluation; and
storing, in the data storage, the medical image determined to be stored.

18. A non-transitory computer readable medium for storing a computer-executable program for functioning a computer as a medical image processing device, the computer-executable program causing the computer to execute:
a process of acquiring a medical image in which a subject is reflected;
a process of performing recognition processing on the medical image;
a process of displaying, on a display, the medical image and a result of the recognition processing of the medical image;
a process of receiving an evaluation related to the result of the recognition processing from a user;
a process of determining whether or not to store the medical image, which is a target of the evaluation, in a data storage based on the evaluation; and
a process of storing, in the data storage, the medical image determined to be stored.
